(19) United States
(12) Patent Application Publication
(10) Pub. No.: US 2016/0166770 A1
(43) Pub. Date: Jun. 16, 2016

(54) Ergonomic Syringe Gripper Apparatus

(71) Applicant: Rafael Emerick Salas, Pembroke Pines, FL (US)

(72) Inventor: Rafael Emerick Salas, Pembroke Pines, FL (US)

(21) Appl. No.: 14/567,024

(22) Filed: Dec. 11, 2014

(51) Int. Cl. A61M 5/31

(52) U.S. Cl. CPC A61M 5/3137; A61M 2005/3139

(57) ABSTRACT

A syringe gripper removably attachable to a syringe cylinder. In one embodiment, the gripper includes an attachment portion defining an opening which is sized and shaped to accept the barrel of a syringe cylinder; a first surface, the first surface for being gripped by a first finger; and a second surface, the second surface for being gripped by a second finger; where the first and second surfaces are substantially coplanar and the first and second surfaces are connected to, and displaced from, the attachment portion. In another embodiment, the invention includes a third surface, the third surface for being gripped by the first finger, and a fourth surface, the fourth surface for being gripped by the second finger. In this embodiment, the third and fourth surfaces are substantially coplanar and the third and fourth surfaces are connected to, but displaced from, the first and second surfaces.

### FIELD OF THE INVENTION

The invention relates generally to the field of medical instruments and more specifically to the field of medical syringes.

### BACKGROUND

Syringes can be very difficult to expunge, especially when filled with viscous fluids. For example, in plastic surgery procedures it is difficult to inject adipose tissue to build or sculpt various areas of the body due to the viscosity of the fat and the relatively small gauge of the needle. Similarly, it is difficult for nurses to inject medications through feeding tubes, again because of the relatively small gauge of the needle.

Further, if the hands of the surgeon or other person using the syringe are small, it is difficult to apply pressure to the plunger of the syringe using the palm of the hand while gripping the extensions of the syringe body with the fingers of the same hand. In addition, during procedures which require the repeated use of a syringe, the current options can be painful and difficult for users who have to repeatedly inject a viscous material multiple times.

Accordingly, there is a need for devices which provide a more comfortable and ergonomic injection motion and which require a shorter grip span for users with small hands. The present invention addresses this need.

### SUMMARY OF INVENTION

The invention relates to a syringe gripper removably attachable to a syringe cylinder. In one embodiment, the gripper includes an attachment portion defining an opening which is sized and shaped to accept the barrel of a syringe cylinder; a first surface, the first surface for being gripped by a first finger; and a second surface, the second surface for being gripped by a second finger; where the first and second surfaces are substantially coplanar and the first and second surfaces are connected to, and displaced from, the attachment portion. In another embodiment, the attachment portion includes a depression sized and shaped to fit extensions of the syringe cylinder. In yet another embodiment, the first and second surfaces are substantially coplanar and the first and second surfaces are connected to, and substantially in the plane of, the attachment portion.

In still yet another embodiment, the gripper is constructed of aluminum or surgical steel. In yet another embodiment, the gripper is constructed of plastic.

In one embodiment, the invention includes a third surface, the third surface for being gripped by the first finger and a fourth surface, the fourth surface for being gripped by the second finger. In this embodiment, the third and fourth surfaces are substantially coplanar and connected to, but displaced from, the first and second surfaces.

### BRIEF DESCRIPTION OF DRAWINGS

The figures are not necessarily to scale, emphasis instead generally being placed upon illustrative principles. The figures are to be considered illustrative in all aspects and are not intended to limit the invention, the scope of which is defined only by the claims.

FIG. 1 is a perspective view of an embodiment of the invention with a syringe positioned in the device.

FIG. 2 is a perspective view of another embodiment of the invention with two locations for the placement of fingers.

FIG. 3 is a side view of an embodiment of the invention of FIGS. 1 and 2 and a syringe shown positioned in the device.

FIG. 4 depicts an embodiment of the device in use.

FIG. 5a is a diagram of another embodiment of the invention including a groove to engage syringe extensions.

FIG. 5b is a side view of the gripper of the invention including a groove.

FIG. 5c is a perspective diagram of the embodiment of FIG. 5a with a syringe positioned within the invention.

### DETAILED DESCRIPTION

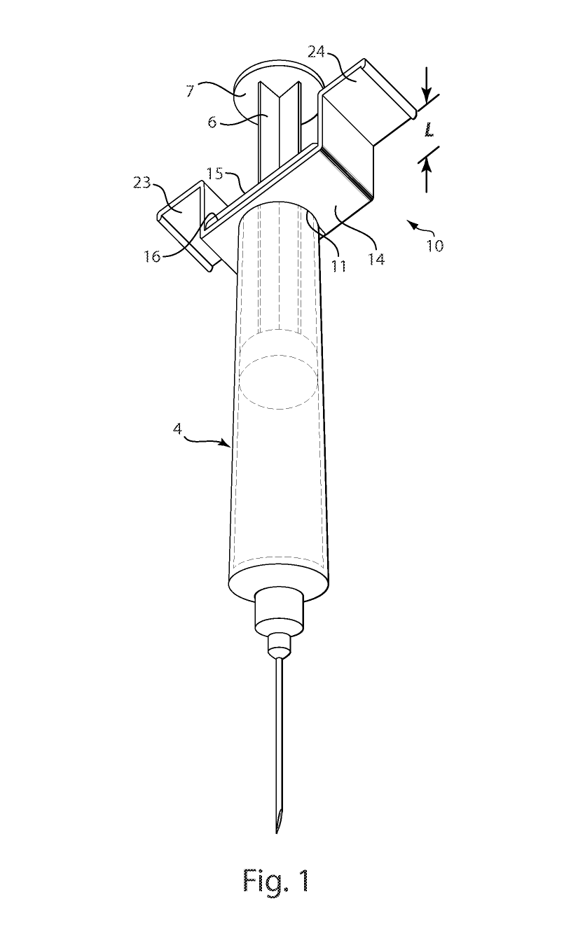

Referring to FIG. 1, the barrel of a syringe cylinder 4 is placed in the opening 11 of the attachment portion 14 of the gripper 10 and, generally, the index finger is placed on the first surface 23 and the middle finger is placed on the second surface 24 (fingers not shown for clarity). The user's palm or thumb is then placed against the end 7 of the plunger 6 of the syringe. Fingers other than the index and middle fingers can be used. The length of the gripper (L) is such that it allows the user's thumb or palm to rest on the end of the plunger 7 when the cylinder 4 is full of liquid. This permits the user to use one hand for injecting the viscous contents of the syringe. Extensions 15 on either side of or surrounding the syringe cylinder body 4, which are normally held by the user's index and middle fingers, instead rest inside a depression in the upper surface 16 of the attachment portion 14 adjacent to the opening 11 to aid in stability. These extensions 15 hold the cylinder body 4 in place as the plunger 6 is forced into the cylinder.

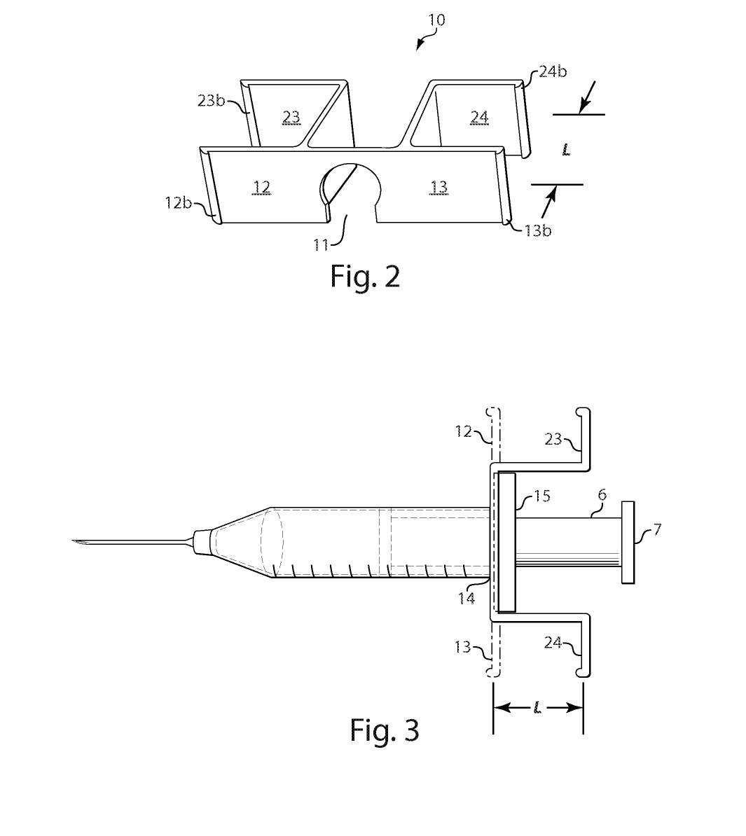

FIG. 2 illustrates a second embodiment which allows for the placement of the index and second fingers in two different positions. A first finger can be placed on the first surface 23 and a second finger can be placed on the second surface 24 as discussed above. Alternatively, a first finger can be placed on the third surface 12 and a second finger can be placed on a fourth surface 13. The first 12 and second 13 surfaces are separated by a distance (L) from, but are parallel to, the third 23 and fourth 24 surfaces. In one embodiment, the first 12 and second 13 surfaces are displaced by an inch to an inch and a half from the third 23 and fourth 24 surfaces. Other displacements are possible. Referring also again to FIG. 1, in each position, the thumb or the palm may be used to depress the plunger 6 of the syringe. The spacing from the end 7 of the plunger 6, when the syringe cylinder is in the filled configuration, to the third 23 and fourth 24 surfaces is less than the distance from the end 7 of the plunger 6 to the first 12 and second 13 surfaces and allows for a user with smaller hands to use one hand to depress the plunger in an ergonomic position, and without discomfort. The embodiment of FIG. 2 allows for comfortable use of the invention by users with hands and fingers of various sizes.

In one embodiment, the first surface 12, second surface 13, third surface 23 and fourth surface 24 provide sufficient surface area for the first and second fingers and so decrease the occurrence of fingers slipping off the surfaces and provide added comfort to the user, including users with fingers of various sizes. In one embodiment, the edges 12b, 13b, of the first 12 and second 13 surfaces respectively, and edges 23b, 24b of the third 23 and fourth 24 surfaces are curved to decrease the possibility of fingers slipping off the edge of the respective surface. In other embodiments, each surface has grooves or raised lines for added grip. In another embodiment, the opening 11 is sized to fit the barrel of different sizes of syringes. As will be appreciated, other dimensions and shape configurations can be used.

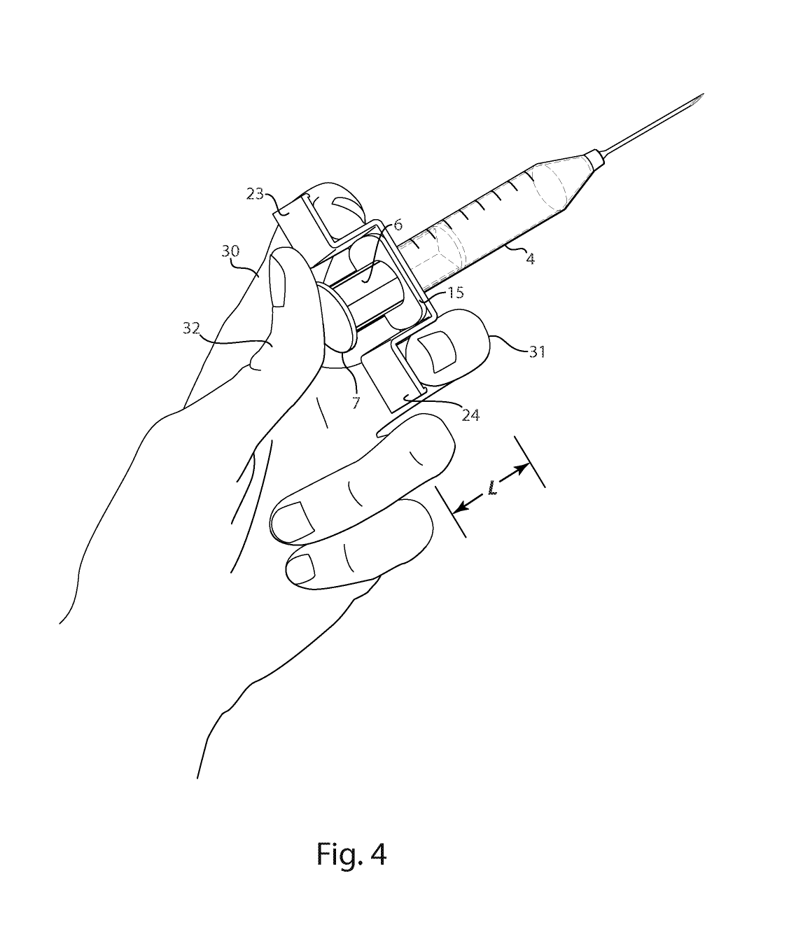

Referring to FIG. 3, the various embodiments, configurations of the gripping device are shown. In one embodiment, the finger surfaces 23 and 24 are shown displaced from the plane of the attachment portion 14. In a second embodiment, the second surfaces 12 and 13 are in the plane of the attachment portion 14 and displaced from the surfaces 23 and 24. In another embodiment, surfaces 12 and 13 are positioned in the plane of the attachment portion 14 and there are no surfaces 23 and 24. This last embodiment allows users with larger fingers who are unable to engage the syringe extensions 16 comfortably to have a larger surface to engage.

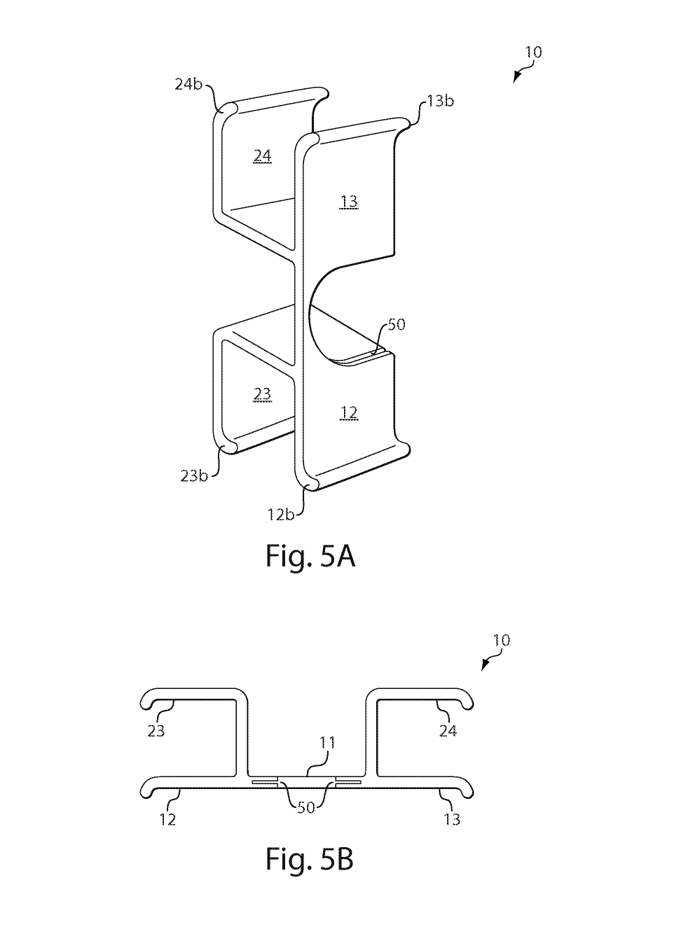

Referring to FIG. 4, an embodiment of the invention of FIG. 1 is shown, with the first 30 and second fingers 31 of a user engaged with surfaces 12 and 13, thumb 32 pressed against the end 7 of the plunger 6, and the barrel of a syringe cylinder 4 engaged within the gripper opening 11. In this embodiment, the invention is placed around the extensions of the syringe 15. As explained above, the length (L) of the gripper 10 is such that it allows the user's thumb or palm to rest on the end 7 of the plunger 6 when the cylinder 4 is full of liquid and the plunger 6 is extended. Also shown in FIG. 3 is the embodiment of FIG. 2 with the lower surfaces shown in phantom. In this embodiment, the placement of the fingers is a distance L further from the attachment surface 14 than in the embodiment of FIG. 1.

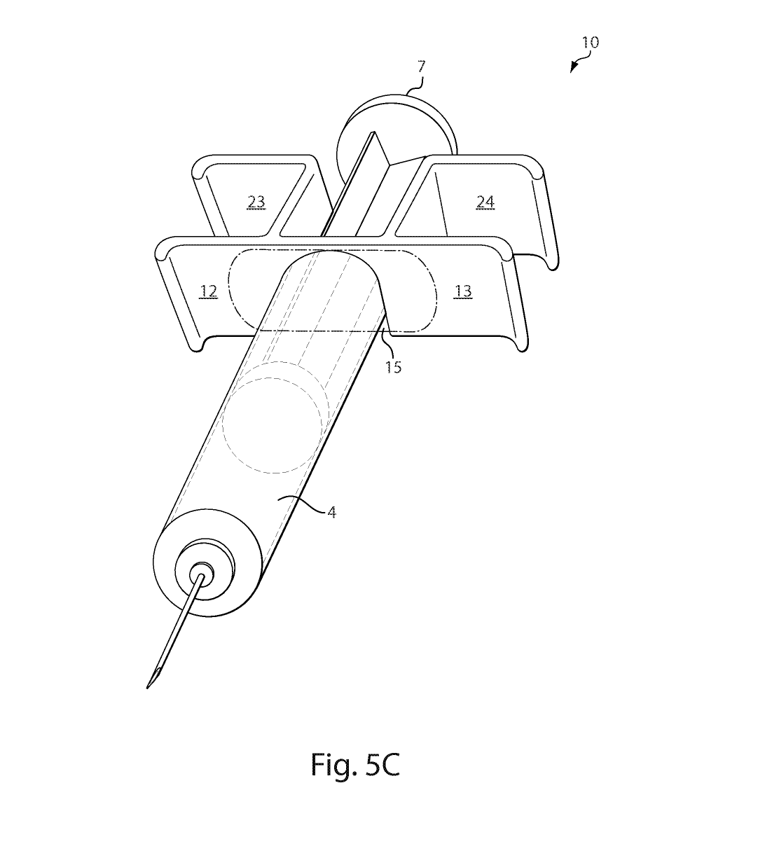

Referring to FIGS. 5a, 5b, and 5c, in another embodiment, the lower surfaces 12 and 13 define a groove 50 into which the extensions 15 of the syringe can be inserted. Normally, with the other embodiments, the extensions 15 of the syringe are pressed against the top surface that defines the opening 11 when the user has his or her thumb on the plunger head 7. The act of putting pressure on the plunger head 7, along with the friction fit of the syringe barrel 4 against the edges of the opening 11, holds the syringe in engagement with the gripper. This new embodiment provides a way to assure that the syringe and the gripper do not separate once the user reduces pressure on the plunger 7. FIG. 5c depicts a syringe 4 positioned with its extensions (shown in phantom 15) located within the gripper.

In one embodiment, the invention is constructed of aluminum or surgical steel. In this embodiment, the invention is re-useable, sturdy and allows for sterilization such as with the use of an autoclave. In another embodiment, the invention is constructed of stiff plastic and may be disposable.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes, and not as construction drawings. Omitted details and modifications or alternative embodiments are within the purview of persons of ordinary skill in the art.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A syringe gripper removably attachable to a syringe cylinder comprising:
an attachment portion defining an opening sized and shaped to accept the barrel of a syringe cylinder;
a first surface, said first surface for being gripped by a first finger; and
a second surface, said second surface for being gripped by a second finger;
the first and second surfaces being substantially coplanar and the first and second surfaces fixedly connected to, and displaced from, said attachment portion.

2. The gripper of claim 1 wherein the attachment portion includes a depression sized and shaped to fit extensions of the syringe cylinder.

3. The gripper of claim 1 wherein the gripper is constructed of aluminum or surgical steel.

4. The gripper of claim 1 wherein the gripper is constructed of plastic.

5. The gripper of claim 1 further comprising:
a third surface, said third surface for being gripped by the first finger;
a fourth surface, said fourth surface for being gripped by the second finger;
wherein the third and fourth surfaces are substantially coplanar and the third and fourth surfaces are also coplanar with, and fixedly attached to, the attachment portion.

6. The gripper of claim 1 wherein the attachment portion defines a horizontal groove into which the extensions of the syringe can be engaged.

7. (canceled)
